(19) United States
(12) Patent Application Publication
(10) Pub. No.: US 2025/0145623 A1
(43) Pub. Date: May 8, 2025

(54) METHODS OF PREPARATION OF HETEROCYCLIC COMPOUNDS

(71) Applicant: HOTSPOT THERAPEUTICS, INC., Boston, MA (US)

(72) Inventors: Jon P. LAWSON, Wildwood, MO (US); Ken CARSON, Princeton, NJ (US); Yingzhi BI, Plainsboro, NJ (US)

(21) Appl. No.: 19/016,575

(22) Filed: Jan. 10, 2025

Related U.S. Application Data
(63) Continuation of application No. PCT/US2023/070059, filed on Jul. 12, 2023.
(60) Provisional application No. 63/388,344, filed on Jul. 12, 2022.

(51) Int. Cl. C07D 471/04; C07D 213/48; C07D 213/61; C07D 249/08; C07D 249/12; C07D 401/06

(52) U.S. Cl. CPC C07D 471/04; C07D 213/48; C07D 213/61; C07D 249/08; C07D 249/12; C07D 401/06

(57) ABSTRACT

Provided herein are methods of preparation of compound 101.

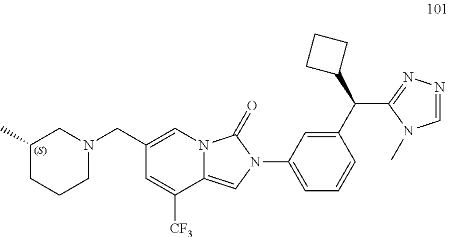

Also provided are intermediate compounds useful in the preparation of compound 101, and methods of preparation of those intermediate compounds.

### CROSS-REFERENCE TO RELATED APPLICATIONS

This application is a continuation of International (PCT) Patent Application No. PCT/US2023/070059, filed on Jul. 12, 2023, which claims the benefit of, and priority to, U.S. Patent Application No. 63/388,344, filed on Jul. 12, 2022, the content of each of which is hereby incorporated by reference in its entirety.

### BACKGROUND

Cbl-b is a E3 ubiquitin-protein ligase that functions as a negative regulator of T-cell activation. Modulation of Cbl-b has been shown to be a therapeutic target for a diseases and disorders. There remains a need for efficient means of preparing compounds that inhibit Cbl-b.

### SUMMARY

In an aspect, provided herein is a method of producing compound 101, the method comprising the step:
a) reacting compound 19 with X—C(O)—Y, wherein X and Y are each leaving groups, to produce compound 101

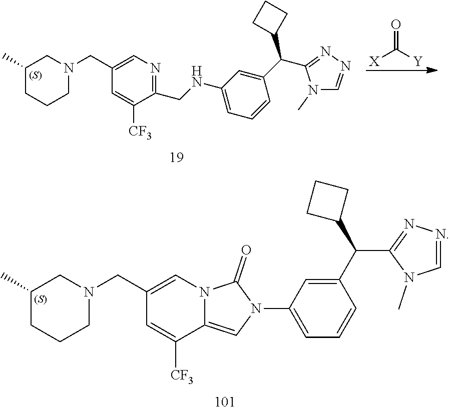

In another aspect, provided is an intermediate compound useful for the preparation of compound 101

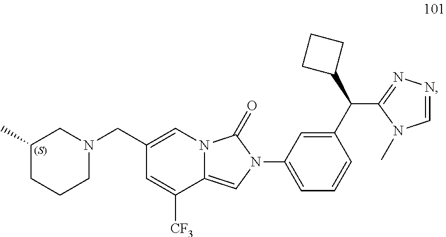

wherein the intermediate compound is selected from the group consisting of compounds 1-19 of schemes 1-3.

In yet another aspect, provided herein is a method of producing an intermediate compound useful for the preparation of compound 101

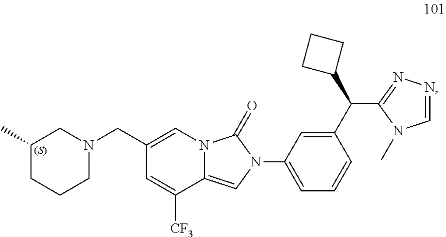

wherein the intermediate compound is selected from the group consisting of compounds 1-19 of schemes 1-3, the method comprising steps found in the examples.

In yet another aspect, provided herein is a method of synthesis of compounds 1-19 of schemes 1-3.

In yet another aspect, provided is a method of producing compound 8 with structure shown below, the method comprising the steps:
e) separating the racemic mixture 6 by chiral Supercritical fluid chromatography (SFC) to obtain compound 7
and

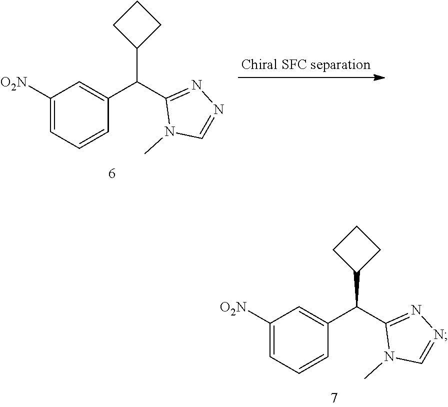 [SMILES: Cn1cnnc1C(c1cccc([N+](=O)[O-])c1)C1CCC1 ; Cn1cnnc1[C@@H](c1cccc([N+](=O)[O-])c1)C1CCC1]

f) reducing compound 7 to provide compound 8

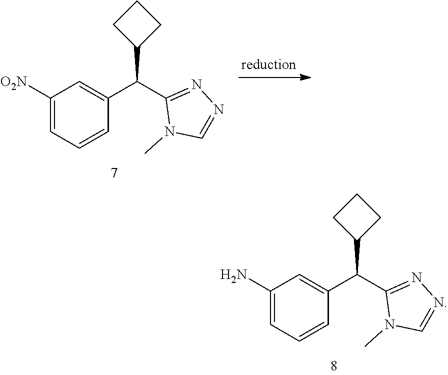 [SMILES: Cn1cnnc1[C@@H](c1cccc([N+](=O)[O-])c1)C1CCC1 ; Cn1cnnc1[C@@H](c1cccc(N)c1)C1CCC1]

### DETAILED DESCRIPTION

The disclosure provides methods of synthesis of a compound referred to herein as compound 101, having the structure shown below:

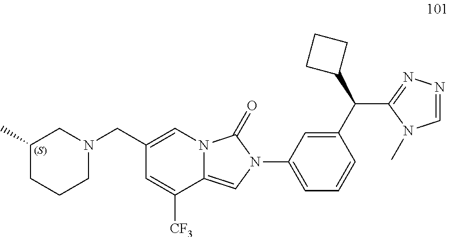

or a pharmaceutically acceptable salt thereof. Also provided are intermediates useful in the synthesis of compound 101 and methods of synthesis of those intermediates.
Compound 101 is an inhibitor of Cbl-b and has potential use as an agent for the treatment of certain diseases, including cancer.

In an aspect, provided herein is a method of producing compound 101, the method comprising the step:
a) reacting compound 19 with X—C(O)—Y, wherein X and Y are each leaving groups, to produce compound 101

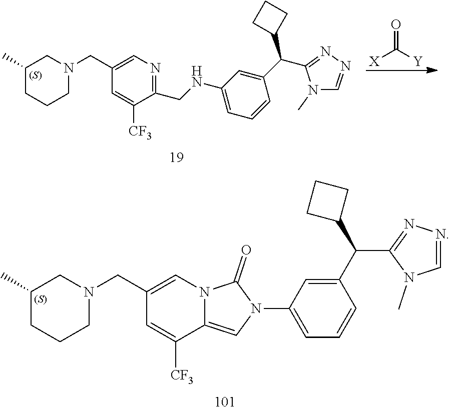

In some embodiments X and Y are each selected independently from Cl, Br, I, OCCl3, imidazolyl radical and p-nitrophenoxy radical.

In some embodiments, X—C(O)—Y is phosgene, bis(trichloromethyl)carbonate, p-nitrophenylchloroformate, or carbonyldiimidazole. In some embodiments, X—C(O)—Y is phosgene or bis(trichloromethyl)carbonate. In some embodiments, X—C(O)—Y is bis(trichloromethyl)carbonate.

In some embodiments, the method comprises the step:
a) reacting compound 19 with bis(trichloromethyl)carbonate (BTC), to produce compound 101

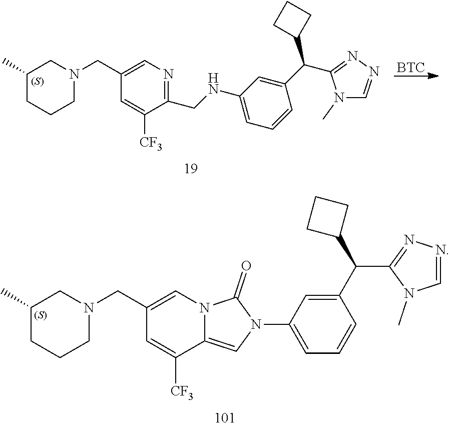

In some embodiments, the method further comprises the step:
b) reacting compound 18 with compound 8 in the presence of a reducing agent to produce compound 19

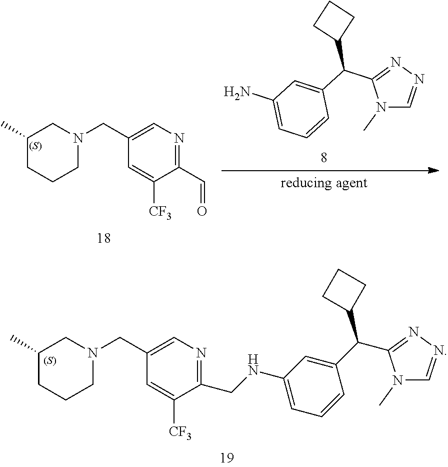

In some embodiments, the step b) reducing agent is a borohydride, such as NaBH(OAc)₃ or NaBH₃CN.

In some embodiments, in step b) compounds 18 and 8 are first reacted to remove water (such as with a Dean-Stark trap) and give compound 18′

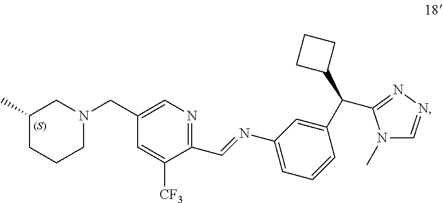

prior to contact with the reducing agent, and the reducing agent is then reacted with 18′ to give compound 19.

In some embodiments, the method comprises the step:
b) reacting compound 18 with compound 8 in the presence of NaBH(OAc)₃ to produce compound 19

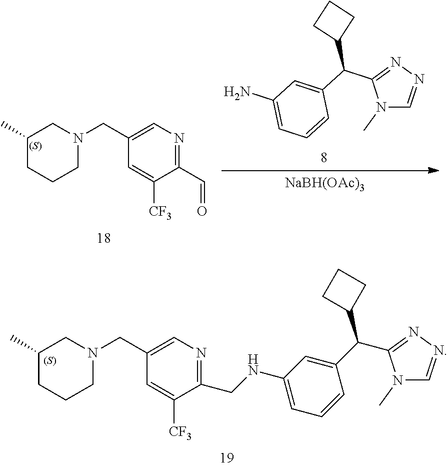

In some embodiments, the method further comprises the steps:
c) reacting compound 14′ with compound 11 in the presence of a catalyst A to produce compound 15′

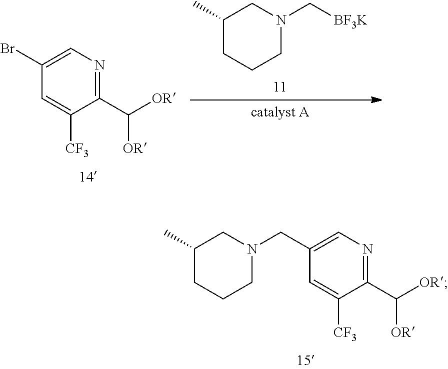

and
d) reacting compound 15′ with acid to produce compound 18

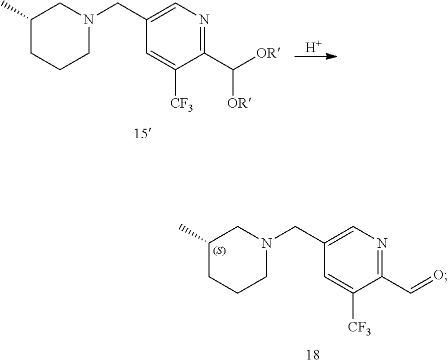

wherein each R′ is independently C₁₋₆ alkyl; or
both R′ groups are taken together as a C₁₋₆ alkylene; and
catalyst A is an organometallic catalyst.

In some embodiments, each R′ is methyl or ethyl, or both R′ groups taken together form —CH₂—CH₂— or —CH₂CH₂CH₂—. In some embodiments, both R′ groups taken together form —CH₂—CH₂—.

In some embodiments, catalyst A comprises a palladium atom, preferably wherein catalyst A is suitable for catalyzing a Suzuki reaction. In some embodiments, catalyst A is formed from the combination of a Pd (II) salt (such as Pd(OAc)₂) and xphos.

In some embodiments, the method further comprises the steps:
c) reacting compound 14 with compound 11 in the presence of a Pd(OAc)2 and xphos to produce compound 15

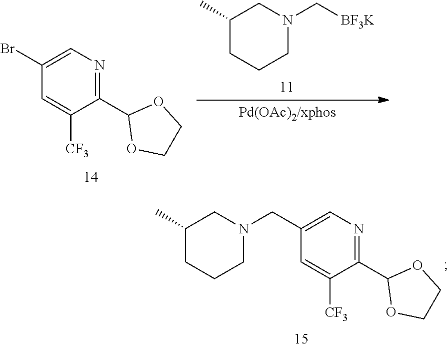

d) reacting compound 15 with acid to produce compound 18

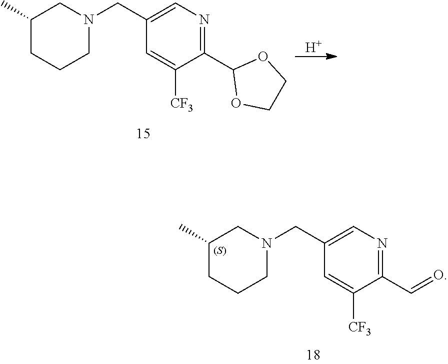

In some embodiments, the method further comprises the steps:
e) separating the racemic mixture 6 by chiral Supercritical fluid chromatography (SFC) to obtain compound 7

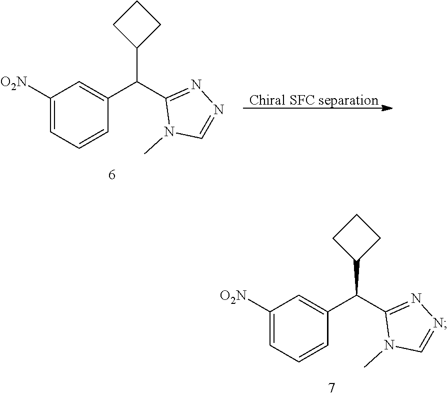 [SMILES: Cn1cnnc1C(c1cccc([N+](=O)[O-])c1)C1CCC1 ; Cn1cnnc1[C@@H](c1cccc([N+](=O)[O-])c1)C1CCC1]

and
f) reducing compound 7 to provide compound 8

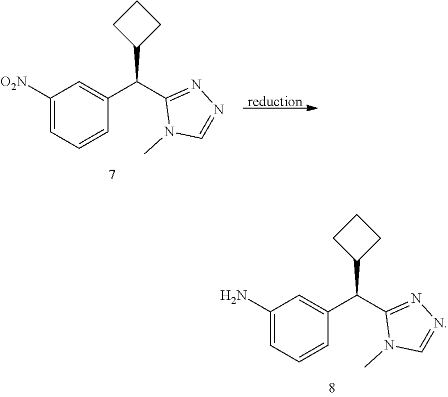 [SMILES: Cn1cnnc1[C@@H](c1cccc([N+](=O)[O-])c1)C1CCC1 ; Cn1cnnc1[C@@H](c1cccc(N)c1)C1CCC1]

In some embodiments, the step e) separating is performed on a cellulose-SC column. In some embodiments the step e) separating is performed with mobile phase A is CO₂, and mobile phase B comprises methanol, acetonitrile, and NH₃. In some embodiments, mobile phase B is methanol:acetonitrile from 2:3 to 3:2 with 1-3 mM NH₃.

In some embodiments, the step f) reducing is performed by catalytic hydrogenation. In some embodiments, the step f) reducing is performed with hydrogen and a catalyst comprising palladium or platinum. In some embodiments the step f) reducing is performed with hydrogen and platinum on carbon (Pt/C).

In some embodiments, the method further comprises the steps:
g) reacting compound 3 with methyl isothiocyanate to obtain compound 4

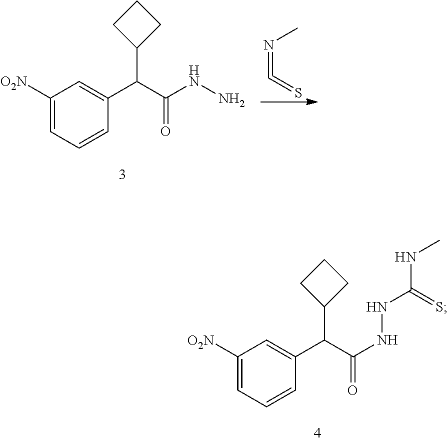

h) reacting compound 4 with base to provide compound 5

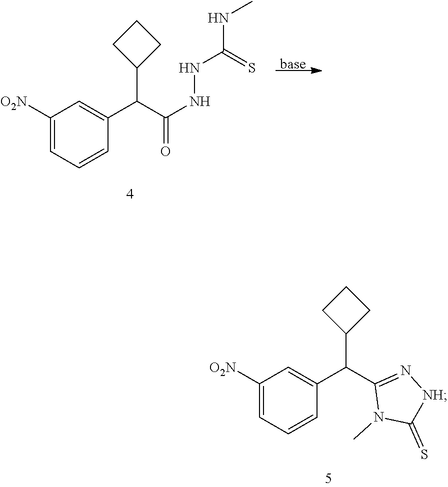 [SMILES: CNC(=S)NNC(=O)C(c1cccc([N+](=O)[O-])c1)C1CCC1 ; Cn1c(C(c2cccc([N+](=O)[O-])c2)C2CCC2)n[nH]c1=S]

and
i) reacting compound 5 with sodium nitrite and acid to produce compound 6

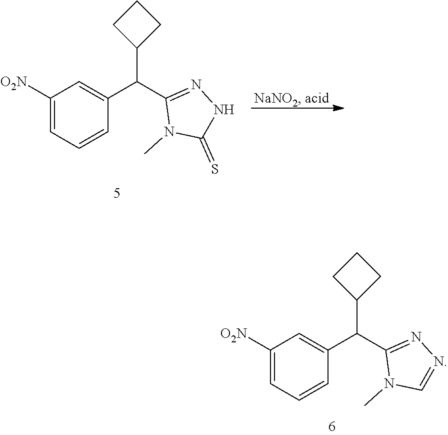 [SMILES: Cn1c(C(c2cccc([N+](=O)[O-])c2)C2CCC2)n[nH]c1=S ; Cn1cnnc1C(c1cccc([N+](=O)[O-])c1)C1CCC1]

In some embodiments, the base in step h) is sodium hydroxide.

In some embodiments, the step i) acid is nitric acid.

In some embodiments, the method further comprises the steps:
g) reacting compound 3 with methyl isothiocyanate to obtain compound 4

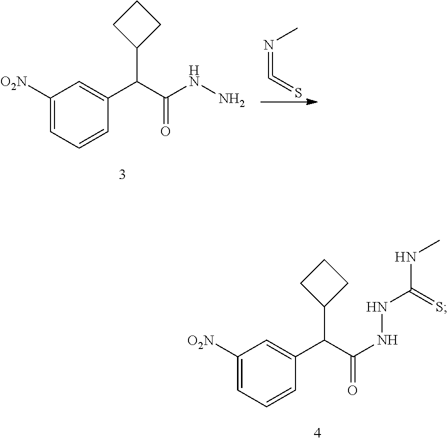

h) reacting compound 4 with base to provide compound 5

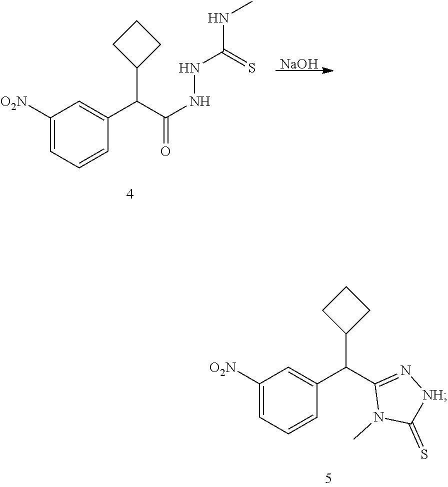

and
i) reacting compound 5 with sodium nitrite and acid to produce compound 6

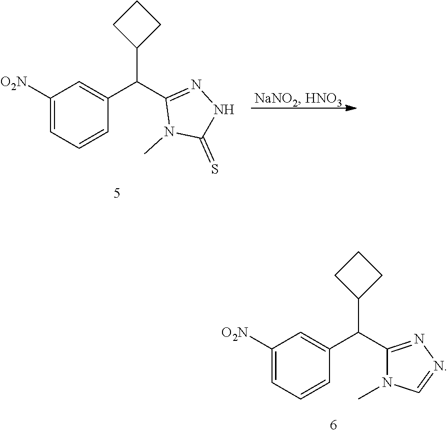 [SMILES: Cn1c(C(c2cccc([N+](=O)[O-])c2)C2CCC2)n[nH]c1=S ; Cn1cnnc1C(c1cccc([N+](=O)[O-])c1)C1CCC1]

In some embodiments, compound 101 is crystallized from a mixture of isopropyl acetate and heptane.

In some embodiments, compound 101 is at least 95% pure by HPLC.

In another aspect, provided is an intermediate compound useful for the preparation of compound 101

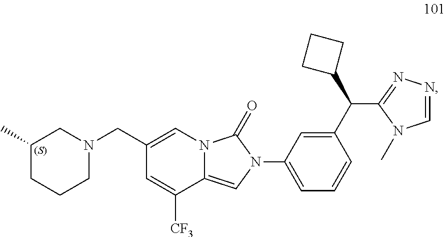

wherein the intermediate compound is selected from the group consisting of compounds 1-19 of schemes 1-3.

In some embodiments, the intermediate compound has the structure

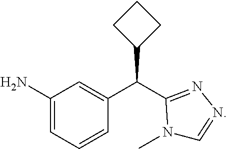 [SMILES: Cn1cnnc1[C@@H](c1cccc(N)c1)C1CCC1]

In some embodiments, the intermediate compound has the structure

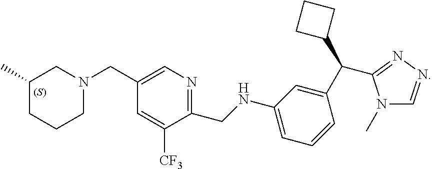

In yet another aspect, provided herein is a method of producing compound 8 with structure shown below, the method comprising the steps:
e) separating the racemic mixture 6 by chiral Supercritical fluid chromatography (SFC) to obtain compound 7

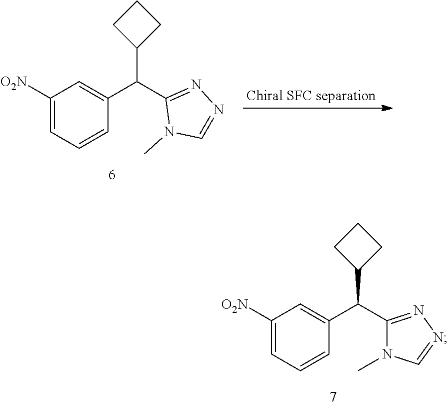 [SMILES: Cn1cnnc1C(c1cccc([N+](=O)[O-])c1)C1CCC1 ; Cn1cnnc1[C@@H](c1cccc([N+](=O)[O-])c1)C1CCC1]

and
f) reducing compound 7 to provide compound 8

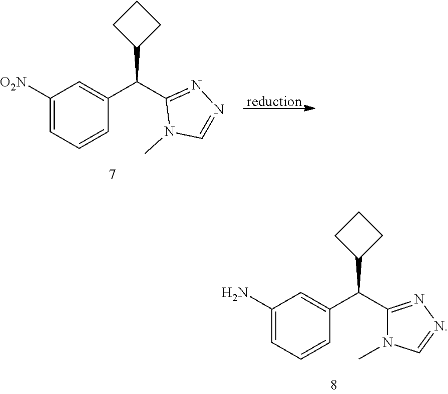 [SMILES: Cn1cnnc1[C@@H](c1cccc([N+](=O)[O-])c1)C1CCC1 ; Cn1cnnc1[C@@H](c1cccc(N)c1)C1CCC1]

In some embodiments of the method of producing compound 8, the step e) separating is performed on a cellulose-SC column.

In some embodiments of the method of producing compound 8, the step e) separating is performed with mobile phase A is CO₂, and mobile phase B comprises methanol, acetonitrile, and NH₃.

In some embodiments of the method of producing compound 8, the mobile phase B is methanol:acetonitrile from 2:3 to 3:2 with 1-3 mM NH₃.

In some embodiments of the method of producing compound 8, the step f) reducing is performed by catalytic hydrogenation, such as with hydrogen and a catalyst comprising palladium or platinum.

In some embodiments of the method of producing compound 8, the method further comprises the steps:
g) reacting compound 3 with methyl isothiocyanate to obtain compound 4

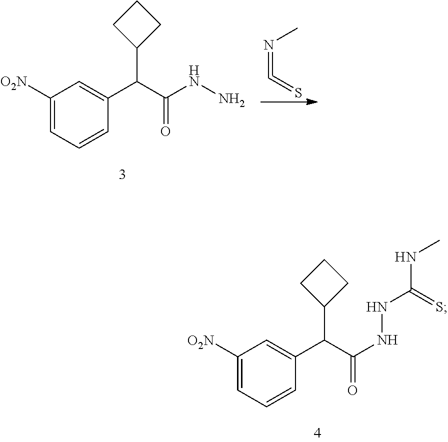

h) reacting compound 4 with base to provide compound 5

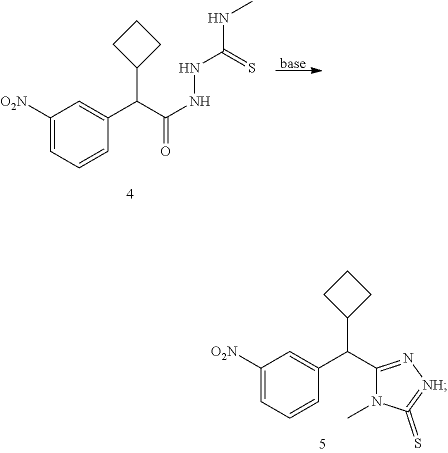

and
i) reacting compound 5 with sodium nitrite and acid to produce compound 6

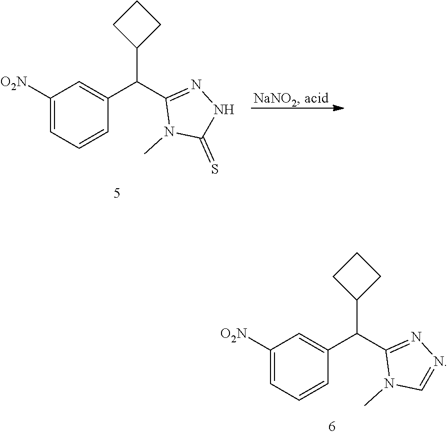 [SMILES: Cn1c(C(c2cccc([N+](=O)[O-])c2)C2CCC2)n[nH]c1=S ; Cn1cnnc1C(c1cccc([N+](=O)[O-])c1)C1CCC1]

In yet another aspect, provided herein is a method of synthesis of compounds 1-19 of schemes 1-3.

### Definitions

The term “alkyl” refers to a straight or branched alkyl group. “C₁₋₆ alkyl” refers to an alkyl group of from 1-6 carbons. Exemplary alkyl groups are methyl, ethyl, propyl, isopropyl, butyl, isobutyl, and tert-butyl.

The term “alkylene” refers to a straight or branched hydrocarbon group which is bivalent. “C₁₋₆ alkylene” refers to an alkylene group of from 1-6 carbons. Exemplary alkylene groups are —CH₂CH₂—, —CH₂CH₂CH₂—, and —CH₂CH(CH₃)CH₂—,

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

A “reducing agent” as used herein refers to a chemical composition or combination thereof which is able to effect a chemical reduction of a substrate from an oxidized form to a reduced form. Reducing agents include hydride-based reagents, including aluminum hydrides such as lithium aluminum hydride and diisobutylaluminum hydride, and borohydrides such as sodium borohydride, NaBH(OAc)₃, and NaBH₃CN. Reducing agents especially suitable for reductive amination reactions include NaBH(OAc)₃ and NaBH₃CN. Reducing agents can also include agents for catalytic hydrogenation.

“Catalytic hydrogenation” as used herein is a reaction in which a chemical reduction of a substrate is performed by the use of hydrogen gas and a catalyst. The catalyst may comprise a metal atom, such as Pd, Pt, or Ni, which may be charged or neutral. The catalyst may comprise a salt of the metal atm or a coordination complex of the metal atom with a non-metallic ligand.

Examples of catalysts for catalytic hydrogenation include Pd on carbon, Pt on Carbon, and Raney Ni.

A “leaving group” as used herein refers to a chemical group which can easily break a chemical bond that attaches them to a molecule and leave with the electrons contained in that bond. Leaving groups can be described as radicals (i.e., they are attached to a molecule by a chemical bond, and that bond is not part of the leaving group) but often leave as anions. Examples of leaving groups include halogens Cl, Br, and I, alkoxys such as trichloromethoxy, phenoxys such as p-nitrophenoxy, and other groups which can stabilize an anion such as imidazolyl.

A “Suzuki reaction” is a chemical coupling reaction between an organic group containing a halogen or certain other leaving groups, and a molecule including a boron group such as a boronic acid or boron salt. Catalysts suitable for the Suzuki reaction include various organometallic catalysts, such as those comprising palladium and an organic ligand such as xphos. Numerous catalysts and substrates are known. (see, for example, R. Martin and S. Buchwald, Acc. Chem. Res., 2008, 41(11): 1461-1473).

### Examples

The invention, now being generally described, will be more readily understood by reference to the following examples, which are included merely for purposes of illustration of certain aspects and embodiments of the present invention, and are not intended to limit the invention.

### Abbreviations

| ABBREVIATION | DEFINITION |
|---|---|
| ACN | Acetonitrile |
| BTC | Bis(trichloromethyl)carbonate |
| DCM | Dichloromethane |
| DMF | N,N-dimethylformamide |
| EA | Ethyl acetate |
| EtOH | Ethanol |
| ¹H-NMR | Proton nuclear magnetic resonance |
| HPLC | High performance liquid chromatography |
| IPC | In process control |
| KF | Karl Fischer assay (for water content) |
| LOD | Loss on drying |
| MeOH | Methanol |
| MTBE | Methyl t-butyl ether |
| NFM | N-formylmorpholine |
| p-TSA | Para-toluenesulfonic acid |
| R.T. | Room temperature |
| SFC | Supercritical fluid chromatography |
| THF | Tetrahydrofuran |
| Xphos | 2-(Dicyclohexylphosphino)-2′,4′,6′- |
| | triisopropyl-1,1′-biphenyl |

Reaction temperatures are reported as internal temperatures unless otherwise specified. Chemical intermediates, reagents, and solvents were obtained from commercial sources.

NMR: ¹H spectra were recorded on a 300 MHz or higher spectrometer. Chemical shifts were referenced to residual solvent signals at δ 2.50 (DMSO-d6) relative to TMS as internal standard wherever applied.

HPLC for chemical purity and chiral purity measurement were as detailed in Example 1.

### Example 1. HPLC Procedures

TABLE A
General HPLC Purity method and IPC
method for compounds 1-7 and 12.

| Column | YMC Triart Phenyl (4.6*150 mm, 3 um) | |
|---|---|---|
| Mobile phase | MPA: 0.05% TFA in water | |
| | MPB: 0.05% TFA in ACN | |
| Column temperature | 30° | C. |
| Detector | 205 | nm |
| Flow rate | 1.2 | mL/min |
| Run time | 20.1 | minutes |
| Dilution | MeOH | |
| Injection volume | 5 | uL |
| Post run | 5 | min |

| | | | | | | |
|---|---|---|---|---|---|---|
| Gradient | Time (min) | 0.0 | 10 | 18 | 20 | 20.1 |
| | MPA % | 70 | 45 | 20 | 20 | 70 |
| | MPB % | 30 | 55 | 80 | 80 | 30 |

TABLE B
HPLC IPC method and HPLC purity method for compounds 15-18.

| Column | XDB C18(4.6*50 mm, 1.8 um) | | | | |
|---|---|---|---|---|---|
| Mobile phase | MPA: 0.05% TFA in water | | | | |
| | MPB: 0.05% TFA in ACN | | | | |
| Column temperature | 40° C. | | | | |
| Detector | 220 nm | | | | |
| Flow rate | 1.2 mL/min | | | | |
| Run time | 6.6 minutes | | | | |
| Dilution | ACN | | | | |
| Injection volume | 5 uL | | | | |
| Post run | 2 min | | | | |
| Gradient | Time (min) | 0.0 | 4.5 | 6.5 | 6.6 |
| | MPA % | 90 | 10 | 10 | 90 |
| | MPB % | 10 | 90 | 90 | 10 |

TABLE C
HPLC IPC method for HOS-15867-24, 0 and
HPLC purity method for compound 19

| Column | YMC Triart Phenyl (4.6*150 mm, 3 um) | |
|---|---|---|
| Mobile phase | MPA: 0.05% TFA in water | |
| | MPB: 0.05% TFA in ACN | |
| Column temperature | 30° | C. |
| Detector | 245 | nm |
| Flow rate | 1.2 | mL/min |
| Run time | 20.1 | minutes |
| Dilution | ACN | |
| Injection volume | 5 | uL |
| Post run | 5 | min |

| | | | | | | |
|---|---|---|---|---|---|---|
| Gradient | Time (min) | 0.0 | 10 | 18 | 20 | 20.1 |
| | MPA % | 70 | 45 | 20 | 20 | 70 |
| | MPB % | 30 | 55 | 80 | 80 | 30 |

TABLE D
HPLC purity method for compound 101

| Column | ACE Excel 2 C18-AR(4.6*150 mm, 3 um) | |
|---|---|---|
| Mobile phase | MPA: 10 mmol NH₄OAC in water | |
| | MPB: MEOH | |
| Column temperature | 40° | C. |
| Detector | 245 | nm |
| Flow rate | 0.4 | mL/min |
| Run time | 40 | minutes |
| Dilution | ACN | |
| Injection volume | 5 | uL |
| Post run | 5 | min |

| | | | | | | |
|---|---|---|---|---|---|---|
| Gradient | Time (min) | 0.0 | 15 | 30 | 35 | 40 |
| | MPA % | 90 | 30 | 25 | 10 | 10 |
| | MPB % | 10 | 60 | 75 | 90 | 90 |

TABLE E
Chiral purity method for (S)-3-Methylpiperidine
hydrochloride (compound 10):

| | |
|---|---|
| Column | YMC Amylose-C (4.6*250 mm, 5 um) |
| Mobile phase | Hex:EtOH = 200:1 |
| Column temperature | 40° C. |
| Detector | 205 nm |
| Flow rate | 0.7 mL/min |
| Run time | 30 minutes |
| Dilution | Hex:EtOH = 200:1 |
| Injection volume | 2 uL |
| Post run | 0 min |

TABLE F
Chiral purity method for compound 15.

| | |
|---|---|
| Column | OJ-H (4.6*250 mm, 5 um) |
| Mobile phase | Hex:EtOH = 50:1 |
| Column temperature | 40° C. |
| Detector | 205 nm |
| Flow rate | 1.0 mL/min |
| Run time | 30 minutes |
| Dilution | EtOH |
| Injection volume | 5 uL |
| Post run | 0 min |

TABLE G
Chiral purity method for compound 101

| | |
|---|---|
| Column | IA (4.6*250 mm, 5 um) |
| Mobile phase | Hex:EtOH = 85:15 |
| Column temperature | 40° C. |
| Detector | 245 nm |
| Flow rate | 1.0 mL/min |
| Run time | 30 minutes |
| Dilution | EtOH |
| Injection volume | 5 uL |
| Post run | 0 min |

### Example 2. Synthesis of Compound 8

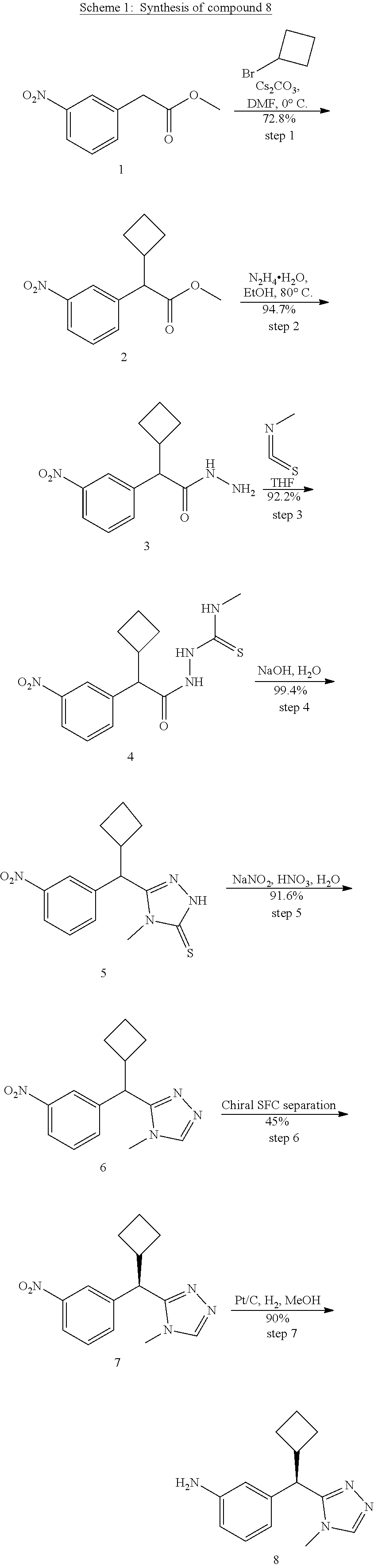

### Synthesis of Compound 2

Under nitrogen atmosphere, DMF (5.0 v) was charged into the reactor. The reaction was stirred for at least 5 minutes. A sample was taken for KF and criterion was KF≤0.1%. Cs₂CO₃ (3.0 eq.; 118 kg) was charged into the reactor. Reaction was nitrogen purged for at least 1.0 hour. The methyl 2-(3-nitrophenyl) acetate (compound 1; 1.0 eq.; 23.6 kg) was added dropwise into the reactor at 20±5° C. and was stirred for at least 1.0 hour. Cyclobutyl bromide (2.0 eq.; 32.3 kg) was added dropwise into the reactor at 20±5° C. The temperature was adjusted to 45±5° C. and the reaction was stirred for at least 16.0 hours at 45±5° C. A sample was taken for HPLC for every 4.0 hours until the area % of compound 1≤5%. IPC=86.5%.

The reaction was cooled to 20±5° C. and MTBE (5.0 v) was charged into the reactor, then the reaction was stirred for at least 30 minutes at 20±5° C. The reaction was filtered and the cake was washed with MTBE (15.0 v) once. The mother liquor was combined.

Organic phase assay: 25.4 kg, 84.5%. Filter cake assay: 0.04 kg, lost: 0.13%.

The mother liquor was transferred to a reactor. Cooled to 5±5° C. and 1N hydrochloric acid (2.0 v) was charged into the reactor. The reaction was stirred for at least 30 minutes at 5±5° C., and then held for at least 30 minutes. Phases were separated, organic phase was collected.

Aqueous phase assay: 0.31 kg, lost: 1.0%.

The organic phase was transferred to a reactor and the jacket temperature was kept at no more than 35° C., and the organic phase was concentrated to 1-2 v. MTBE (2.5 v) was charged into the reactor, then n-Heptane (7.5 v) was charged into the reactor and the reaction was stirred for at least 30 minutes at 20±5° C., then held for at least 30 minutes. Phases were separated, organic phase was collected. The organic phases were combined and transferred to the reactor.

Soft water (5.0 v) was charged into the reactor and stirred for at least 30 minutes at 20±5° C., then held for at least 30 minutes. Phases were separated, organic phase was collected. Organic phase assay: 25.1 kg, 83.4%. Aqueous phase assay: 0.4 kg, lost: 1.3%.

Soft water (5.0 v) was charged into the reactor and stirred for at least 30 minutes at 20±5° C., then held for at least 30 minutes. Phases were separated, organic phase was collected. Organic phase assay: 24.9 kg, 82.7%. Aqueous phase assay: 0.1 kg, lost: 0.3%.

20% sodium chloride aqueous solution (3.0 v) was charged into the reactor and stirred for at least 30 minutes at 20±5° C., then held for at least 30 minutes. Phases were separated, organic phase was collected. The organic phase was transferred to the reactor. The jacket temperature was kept at no more than 45° C. and the organic phase was concentrated to 2-3 v. The temperature was adjusted to 20±5° C. n-Heptane (3.0 v) was charged into the reactor at 20±5° C. The temperature was adjusted to 40±5° C. and the reaction was stirred until the solid dissolved. Compound 2 seed crystal was charged into the reactor at 38±2° C. and the reaction was stirred until the solid crystallized out. The reaction was cooled slowly to 5±5° C. and stirred for at least 4.0 hours at 5±5° C. The reaction was filtered. Reaction sampled by HPLC. The filter cake was dried at 20±5° C., P≤−0.08 MPa, for at least 16.0 hours. Cake sampled for LOD.

For product compound 2, m=21.9 kg, yield: 72.8%, HPLC purity: 99.3%, LOD: 0.49%. Mother liquor assay: 4.31 kg, lost 14.3%.

### Synthesis of Compound 3

Under nitrogen atmosphere, methanol (7.0 v) was charged into the reactor. Started stirring and compound 2 (1.0 eq.; 21.9 kg) was charged into the reactor. Temperature was adjusted to 45±5° C. and 80% hydrazine hydrate (10.0 eq.; 54.9 kg) was added dropwise into the reactor at 45±5° C. for at least 1.0 hour. Reaction was stirred for at least 16.0 hours at 45±5° C. A sample for HPLC was taken and the reaction was run until the area % of Compound 2 ≤5%. IPC=96.4%.

The reaction was cooled slowly to 45±3° C., and a compound 3 seed crystal was charged into the reactor at 45±3° C., and the reaction was stirred until the solid separated out. The reaction was cooled slowly to 20±5° C. and soft water (7.0 v) was added dropwise the into the reactor at 20±5° C. for at least 1.0 hour. Continued stirring at least 4.0 hours at 20±5° C. The reaction was filtered and the cake was washed with soft water (10.0 v) once. Mother liquor assay: 0.6 kg lost 2.7%. A sample was taken for HPLC. HPLC purity: 98.6%.

The filter cake was dried at 60±5° C., P≤−0.08 MPa, for at least 16.0 hours, a sample was taken for KF at least every 4.0 hours until the KF≤0.2%, at which point drying was stopped. Product compound 3: m=20.7 kg, yield: 94.7%, HPLC purity: 99.4%, KF: 0.02%.

### Synthesis of Compound 4

Under nitrogen atmosphere, THF (10.0 v) was charged into the reactor. Stirring was started and continued for at least 5 minutes. A sample was taken for KF and the criterion was KF≤0.1%. Compound 3 (1.0 eq., 18.72 kg) was charged into the reactor. Methyl isothiocyanate (1.2 eq.; 6.95 kg) was added dropwise into the reactor at 25±5° C. for at least 2.0 hours at 25±5° C. The reaction was stirred for at least 16.0 hours at 25±5° C. A sample was taken for HPLC every 4.0 hours until the area % of compound 3 (1.0 eq.)≤1%. IPC: 96.3%.

Absolute ethanol (5 v) was charged into the reactor at 25±5° C. The jacket temperature was kept at no more than 35° C. and the organic phase was concentrated to 4-6 v. Absolute ethanol (10.0 v) was charged into the reactor at 25±5° C. Temperature was adjusted to 60±5° C. and stirring was continued for at least 1.0 hour. The reaction was cooled to 25±5° C. and stirred for at least 4.0 hours at 25±5° C. The area % of Tetrahydrofuran: 9.1%.

The reaction was filtered and the cake was washed with absolute ethanol (4.0 v) once. Mother liquor assay: 1.0 kg, lost: 3.9%.

A sample was taken for LOD, and a sample was taken for HPLC purity. Product compound 4 was kept in a well-closed container at room temperature. Wet cake: 45.1 kg, LOD: 47.7%, contained 23.6 kg, yield: 92.2%, HPLC purity: 99.2%.

### Synthesis of Compound 5

Under a nitrogen atmosphere, soft water (10 v) was charged into the reactor and stirring was started. Temperature was adjusted to 20±5° C., and sodium hydroxide (1.50 eq.; 4.4 kg) was charged into the reactor at 20±10° C. The reaction was stirred for at least 30 minutes until the solids dissolved. The reaction was cooled to 5±5° C. and the solid of compound 4 (1.00 eq.; 45.1 kg) was charged in batches into the reactor at 5±5° C. The reaction was stirred for at least 4.0 hours at 5±5° C. The temperature was adjusted to 20±5° C. and the reaction was stirred for at least 6.0 hours at 20±5° C. Samples were taken for HPLC until the area % of compound 4≤3%. IPC: 99.4%.

The temperature of the system was adjusted to 10±5° C. 1N hydrochloric acid solution was added dropwise into the reactor at 10±5° C. and the pH value of the reaction system was adjusted to 3-6.

The reaction was filtered and the filter cake was washed with soft water (2.0 v) once. The filter cake was collected. Soft water (10.0 v) was charged into the reactor. Agitation was started and the filter cake was charged into the reactor. The temperature was adjusted to 20±5° C., and the reaction was stirred for at least 2.0 hours at 20±5° C.

The reaction was filtered and the filter cake was washed with soft water (2.0 v). Mother liquor lost: 0%.

The filter cake was collected. A sample was taken for LOD and a sample was taken for HPLC. For the product compound 5; Wet cake: 59.9 kg, LOD: 63.5%, contained 21.9 kg, yield: 98.3%, HPLC purity: 99.6%.

### Synthesis of Compound 6

Under nitrogen atmosphere, dichloromethane (5.0 v) was charged into the reactor, then soft water (5.0 v) was charged into the reactor. Stirring was started and compound 5 (1.0 eq.; 16.5 kg) was charged into the reactor. Sodium nitrite (4.0 eq.) was charged into the reactor. The reaction was cooled to 5±5° C. and the 2N nitric acid aqueous solution (2.0 eq.) was added dropwise into the reactor at 5±5° C. for at least 12.0 hours. The temperature was adjusted to 20±5° C. and the reaction was stirred for at least 6.0 hours at 20±5° C. A sample was taken for HPLC for at least every 4.0 hours until the area % of compound 5 was ≤2%. IPC: 97.5%.

The temperature was adjusted to 20±5° C. 15% sodium carbonate aqueous solution (about 2.1 v) was charged into the reactor at 20±5° C. and the pH value of the reaction system was adjusted to 7-8. The reaction was stirred for at least 30 minutes at 20±5° C., then held for at least 30 minutes. The phases were separated, and the organic phase was collected. Dichloromethane (5.0 v) was charged into the reactor with the aqueous phase and the reaction was stirred for at least 30 minutes at 20±5° C., then held for at least 30 minutes. The phases were separated, and the organic phase was collected. The organic phase was transferred to the reactor. Soft water (4.0 v) was charged into the reactor and stirred for at least 30 minutes at 20±5° C., then held for at least 30 minutes. The phases were separated, and the organic phase was collected. 20% sodium chloride aqueous solution (4.0 v) was charged into the reactor and the reaction was stirred for at least 30 minutes at 20±5° C., then held for at least 30 minutes. The phases were separated, and the organic phase was collected. Aqueous phase loss: 0.1%.

The organic phase was transferred to the reactor. The jacket temperature was kept at no more than 40° C. and the organic phase was concentrated to 3-5 v. Methyl tert-butyl ether (10.0 v) was charged into the reactor at 20±5° C. The jacket temperature was kept at no more than 40° C. and the organic phase was concentrated to 3˜5 v. The reaction was filtered and the filter cake was washed with methyl tert-butyl ether (1.0 v) once. Mother liquor loss: 0.6%.

A sample was taken for LOD and a sample was taken for HPLC purity.

Product compound 6: m=4.93 kg, Yield: 98.3%, HPLC purity: 99.6%.

### Synthesis of Compound 7

Compound 6 (10 kg) enantiomers were separated by SFC chromatography under the following conditions to give compound 7.
Column: CHIRAL ART Cellulose-SC, 7*25 cm, 10 m;
Mobile Phase A: CO₂,
Mobile Phase B: MeOH:ACN=1:1 (2 mM NH₃-MeOH);
Flow rate: 250 mL/min;
Gradient: isocratic 36% B;
Column Temperature (° C.): 35;
Back Pressure (bar): 100;
Wave Length: 220 nm;
Retention time 1 (min): 8.59; Retention time 2 (min): 11.68;

Product compound 7 was obtained (3.8 kg, HPLC purity 99.6%; chiral purity 99.9%)

### Synthesis of Compound 8

MeOH (10 L, 10 v), Pt/C (50 g, 5 wt %), and NH₄VO₃ (21.5 g, 0.05 equiv.) were charged into a 20 L high pressure reactor under N₂ atmosphere at room temperature. Compound 7 (1,000 g, 1.0 eq.) was charged into the reactor under N₂ atmosphere at room temperature. (two other batches were performed, one with 1082 g compound 7 and one with 15 g of compound 7, and the final yield reflects combination of all three batches). The system was purged with N₂ three times, then with H₂ three times, then the reaction system was kept at 20 atm. After addition, the reaction was stirred for 12 h at 55-65° C., the H₂ pressure was kept at 10-20 atm, and samples were taken showing residual compound 7 <1.0%.

The reaction was filtered and the filter cake was washed with MeOH (3*10 v) thrice. All filtrates were combined, and this was combined with two other batches of mother liquor to do the concentration. The combined liquors were then concentrated under vacuum at 40° C. to 1-2 v.

MTBE (20.4 L, 10 v) was charged into the residue and the mixture was stirred for 3 h at room temperature. The mixture was filtered and the filter cake was collected to get compound 8 as white solid. Compound 8 was dried under vacuum for 2 h at 40° C. to obtain 1,818 g white solid. HPLC purity: 97.1%. Ee: 98.58%, Isolated yield: 96%.

### Example 3. Synthesis of Compound 11

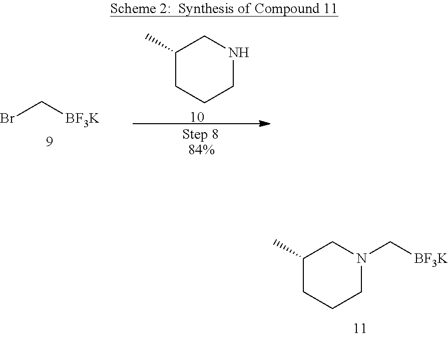

### Synthesis of Compound 11

Under nitrogen atmosphere, tetrahydrofuran (20.0 v) was charged into the reactor. Stirring was started and continued for at least 5 minutes. A sample was taken for KF analysis (test: 0.0143%).

(S)-3-methylpiperidine hydrochloride (Compound 10; 1.0 eq.; 7.00 kg), potassium (bromomethyl) trifluoroborate (compound 9; 1.1 eq.), potassium bicarbonate (2.1 eq.), and potassium iodide (0.1 eq.) were charged into the reactor. The temperature was adjusted to 30±5° C. and the reaction was stirred for at least 1 hour; the temperature was adjusted to 40±5° C. and the reaction was stirred for at least 1 hour; the temperature was adjusted to 50±5° C. and the reaction was stirred for at least 1 hour. The reaction was then heated to 60˜65° C. and the temperature was kept at 60˜65° C. while stirring for at least 6 hours. The reaction was cooled to below 30° C., samples for ¹H-NMR were taken until the chemical shift of 2.6 ppm 2.5 ppm≤0.13:1. (test: 0.05:1).

The temperature was adjusted to 25±5° C. The temperature was kept at 25±5° C., and the reaction was stirred for at least 0.5 hour. The reaction was filtered and the filter cake was washed with THF (2*2 v) twice. All filtrates were combined and concentrated under vacuum to 3˜4 v. Under nitrogen atmosphere, acetone (2*7.0 v) was charged into the reactor at 25±5° C. The inner temperature was kept at no more than 50° C., and the jacket temperature was kept at no more than 70° C. The organic phase was then concentrated to 3˜4 v. Under nitrogen atmosphere, the organic phase was transferred to the reactor through the fluid filter. The inner temperature was kept at no more than 50° C., and the jacket temperature at no more than 70° C. The organic phase was concentrated to 3˜4 v. The organic phase was transferred into a 50 L rotary steaming bottle, and concentrated under vacuum to 1˜2 v. 3 v purified water was charged into the bottle, and the mixture was concentrated to 3 v. 29.4 Kg of aqueous phase was obtained, and was checked by ¹H-NMR, proton 2.3 ppm:2.1 ppm=1:0.39, assay of compound 11: 32.4%, yield: 84.2%

### Example 4. Synthesis of Compound 101

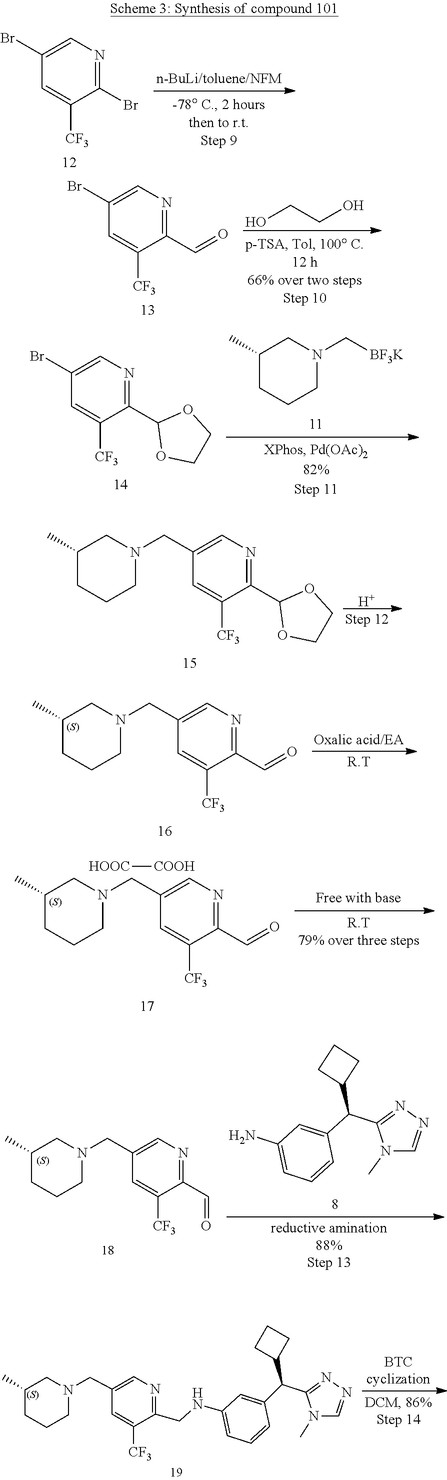

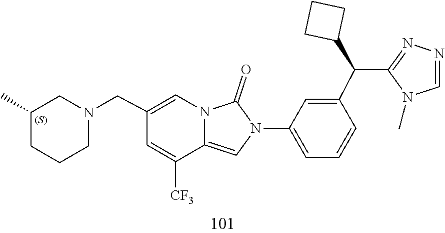

### Synthesis of Compound 14

Under nitrogen atmosphere, toluene (10.0 v) was charged into the reactor, stirred for at least 5 minutes. A sample was taken for KF and criterion is KF≤0.05%. (result: KF=0.003%).

Stirring was started and compound 12 (1.00 eq.; 100 g) was charged into the reactor. The temperature was adjusted to −77±5° C. The temperature was kept at −77±5° C., then n-BuLi (1.10 eq.) was added dropwise into the reactor (preferably added over at least 2 hours) and the reaction was stirred for at least 0.5 hour. Temperature was kept at −77±5° C., a sample was taken for HPLC until the area % of compound 12 ≤4.0%.

The temperature was kept at −77±5° C., and NFM (1.5 eq.; 56.6.g) was added dropwise into the reactor. The temperature was controlled at −77±5° C., sampling was sent to HPLC for testing, until two consecutive samples show purity changes ≤2.0%. (results: HPLC purity changes between two samples ≤0.1%). 1 h HPLC purity: 93.8%, 2 h HPLC purity: 93.8%.

Under N₂ atmosphere, the reaction mass was warmed to 10±20° C., 18.0% citric acid aqueous solution was charged into the reactor to adjust pH=3˜6 (pH=4). Reaction was maintained at 15˜25° C., and stirred for at least 30 min. Reaction was transferred to an Enamel reactor, and stirred for at least 0.5 h, and held for at least 0.5 h. Phases were separated and the aqueous phase and the organic phase were collected. The aqueous phase was extracted with toluene (4.00 V) once at 20±10° C., and the toluene phase was combined with the organic phase. Soft water (5.00 V) was charged into the reactor containing the organic phase at 20±10° C. The reaction was stirred for at least 0.5 h and held for at least 0.5 h. Phases were separated, and the organic phase was collected and held for a 2nd washing. Under N₂ atmosphere, soft water (5.00 V) was charged into the reactor at 20±5° C. The reaction was stirred for at least 0.5 h, and held for at least 0.5 h. Phases were separated and the organic phase was held for a 3rd washing. Under nitrogen atmosphere, sodium chloride aqueous solution was charged into the reactor and the reaction was stirred for at least 30 minutes at 20±5° C., then held for at least 30 minutes. The phases were separated and the organic phase was collected. 1% of material was lost in the aqueous phase.

Under nitrogen atmosphere, the organic phase was transferred to the reactor, the inner temperature was kept at no more than 55° C., or the jacket temperature was kept at no more than 75° C., and the reaction was concentrated to 2.5˜3.5 v. The reaction was cooled to 20˜30° C., and 5 V toluene was charged into the reactor. The reaction was concentrated under vacuum to provide 2.0˜3.0 V (35.65 Kg) of crude compound 13 in solution. HPLC purity: 97.1%. Assay yield: 85%. The crude 13 was then used in the next step for acetal formation.

Under nitrogen atmosphere, the toluene solution of 13, 28.9 Kg toluene, ethylene glycol (5.0 eq.), and p-Toluene sulfonic acid monohydrate (0.05 eq.) were all charged into the reactor, and the reaction was heated to 110˜115° C. The reaction was maintained at 110˜115° C., stirred for at least 12 h, and a sample was taken for HPLC until the area % of compound 13 ≤0.5%. After 16 h IPC, 0.3% residue of compound 13.

The reaction was cooled to 20˜30° C., the phases were separated, the lower phase was extracted with MTBE (0.4 V), and the organic phases were combined. The organic phase was added to a 5% NaHCO₃ aqueous solution (7 V), stirred at 15˜30° C. for 20˜30 min, and allowed to stand for 10˜20 min. the phases were separated, the organic phase was washed with purified water (4 V) twice and brine (4 V) once. There was a 1% loss in the aqueous phase.

The obtained organic phase was mixed with Activated carbon (20 wt %) and stirred at 15˜30° C. for at least 12 h. The mixture was filtered, and the filter cake was washed with toluene (2 V) twice. There was a 1˜2% loss in the filter cake.

The filtrate was transferred into the reactor, and concentrated under vacuum to 1.0˜2.0 v. Heptane (4 v) was charged into the reactor and concentrated to 1.0˜2.0 v twice. Heptane (0.8 V) was charged into the reactor, the mixture was heated to 45˜50° C. and stirred for 2˜3 h. No solid was observed, the product was dissolved in Heptane. The solution was cooled to 15˜30° C. and stirred for 1˜2 h. Solid was formed gradually. The mixture was cooled to −5˜5° C. and stirred for 1˜2 h. The mixture was filtered, and the filter cake was dried under nitrogen at 15˜30° C. After drying, 10.23 Kg of compound 14 as a yellow solid was obtained, HPLC purity: 99.3%, NMR assay: 95.0%. Yield over two steps: 66.9%. 15% loss in the mother liquor.

### Synthesis of Compound 15

Compound 11 (1.5 eq.; 6000 g) aqueous solution, Cs₂CO₃ (3.0 eq.), Pd(OAc)₂ (0.05 eq.), Xphos (0.1 eq.), and 2-MeTHF (7 v) were charged into the reactor. The system was purged with N₂ for 3 min, then a solution of compound 14 (6.0 kg, 1.0 eq.) was added dropwise in 2-MeTHF (1 v) over 1 h at 70-80° C. The reaction was stirred overnight under N₂ atmosphere at 70-80° C. A sample was taken for IPC showing residual compound 14≤0.5%. IPC: no residue of compound 14.

The reaction system was cooled to room temperature, then was filtered and the filter cake was washed with 2-MeTHF (2 v). No material loss in filter cake. All filtrates were combined and purified water (5 v) was added into the filtrate. Phases were separated and the organic phase was washed with 10% brine (5 v). All aqueous phases were combined, 1% loss in seen in aqueous phase. The organic phase was extracted with 5% citric acid (3*30 mL, 3*6 v) three times. All acid phases were combined and washed with 2-MeTHF (5 v). All organic phases were combined, 6.7% loss seen in organic phase. The acid phase was sampled and showed HPLC purity: 99.3%

The acid phase was transferred into the reactor, maintained at T=15˜30° C., and sat. Na₂CO₃ aqueous solution was added to adjust pH=8˜9. The aqueous phase was extracted with 2-MeTHF (2*5 v) two times, and all 2-MeTHF phases were combined. No material loss seen in the aqueous phase. The 2-Me-THF phase was concentrated under vacuum below 40° C. to get compound 15 as brown oil. HPLC purity: 99.4%, NMR assay: 97.0%, yield: 82%. Crude compound 15 was used directly in the next step.

### Synthesis of Compound 17

H₂O (5 v) was charged into the reactor under N₂ atmosphere at room temperature and stirring was started. The reaction was cooled to 0-10° C. and con. H₂SO₄ (1 v) and compound 15 (5.46 Kg, 1.0 eq.) were charged into the reactor. The reaction was stirred for 16 h at 60° C. A sample was taken to IPC. Test found 3.3% residue of compound 15.

The reaction was cooled to −5˜5° C. and the pH value of the system was adjusted to 8-9 with 12% NaOH aqueous solution slowly (˜12 v) at −5˜5° C. 2-MeTHF (5 v) was added to the reaction, the reaction was warmed to 15˜30° C. and stirred for 30 min. Some of the Na₂SO₄ was not dissolved in the aqueous phase. The reaction was filtered, the filtrate was transferred into the reactor, and the phases were separated. The filter cake was washed with 2-MeTHF (5 v) and the aqueous phase was extracted as above. 0.2% loss in water phase, no loss in filter cake. The 2-MeTHF phases from the two phase separations were combined. The 2-MeTHF phase was concentrated under vacuum below 50° C. to 1-2 v. The residual 2-MeTHF was exchanged with EA (5 v) once. The residue was dissolved in EA (10 v), and the EA solution was charged into the reactor, then anhydrous Oxalic acid (2.0 eq.) was added into the system to form a salt at 15˜30° C. KF of EA solution: 0.0144%. The reaction mixture was stirred for 2 h at 15˜30° C. The mother liquid was sampled to IPC showing residual compound 16. No loss in ML. The filter cake was filtered and washed with EA (2 v) once. The filter cake was collected and dried under N₂ flow to get compound 17 as light yellow solid. 7.9 Kg light yellow solid, HPLC purity: 97.0%, LOD: 0.22, yield 6.16 kg.

### Synthesis of Compound 18

H₂O (15.6 L, 5 v) and compound 18 (3.12 kg) were charged into a 50 L reactor under N₂ atmosphere at room temperature. The reaction was stirred for 30 min at room temperature. The pH value of the system was adjusted with sat. 5% Na₂CO₃ aqueous solution to 8˜9. (8˜9 v). The aqueous phase was extracted with 2-MeTHF (2*15.6 L, 2*5 v) two times, and all 2-MeTHF phases were combined. The 2-MeTHF phase was separated and washed with water (15.6 L, 5 v) once. The 2-MeTHF phase was separated and washed with 10% brine (6.24 L, 2 v) once. The 2-MeTHF phase was concentrated under vacuum below 40° C. to get compound 18 (2.2 kg) as brown oil. KF: 0.104%. yield: 92.8%.

### Synthesis of Compound 19

Compound 8 (1,737.4 g, 1.0 eq.), Compound 18 (2,463.8 g, 1.2 eq.), TsOH (0.05 eq.) and 2-MeTHF (20 v) were charged into 50 L reactor under N₂ atmosphere at 20° C. The reaction was refluxed (85-90° C.) for 10 h under N₂ atmosphere and water was removed by Dean-Stark trap. Samples were taken to IPC by ¹H-NMR (d6-DMSO) showing the peaks in 8.91 ppm:6.41 ppm>1:0.06

The reaction solvent was concentrated under vacuum at 40-50° C. to 3-4 v and exchanged 2-MeTHF with DCM (17.3 L, 10 v). The mixture was then concentrated to 3-4 v. DCM (22.5 L, 13 v), was charged into the reaction, and the reaction was stirred for 0.5 hours. The reaction mixture was then cooled to 5° C.

NaBH(OAc)₃ (3,048 g, 2.0 eq.) was charged in 4 portions into the residual mixture at 0-10° C. for 2 h. The reaction was stirred 2 h at 5° C. and then the temperature was warmed to 20° C. The reaction was stirred overnight and sampled to IPC showing residual compound 8≤3%. The reaction was quenched by the reaction system being added to H₂O (17.4 L, 10 v) at 30° C. and stirred for 2.0 h. The organic phase was separated and washed with H₂O (1*17.4 L, 1*10 v) once. The organic phase was separated and washed with 4% Na₂CO₃ aqueous (1*17.4 L, 1*10 v) once. The organic phase was separated and washed with brine (1*17.4 L, 1*10 v) once. The organic phase was concentrated under vacuum at 40-50° C. to 2-3 v and exchanged DCM with MTBE (3*17.4 L, 3*10 v) three times. The residue was slurried with MTBE (5 v) for 2.0 h at r.t (20±5° C.). The reaction was filtered and the filter cake was collected to get compound 19 as 3103 g solid, HPLC purity: 97.4%; recovered 135 g solid from mother liquid, HPLC purity: 95.2%, Total yield: 88.1%.

### Synthesis of Compound 101

Compound 19 (1.0 eq.) and DCM (7 v) were charged into reactor A under N₂ atmosphere at room temperature. The reaction was kept at 25° C. and then 1-methyl-1H-imidazole (3.0 eq.) was charged to the reactor, and the reaction was stirred for 0.5 h at 25° C. The mixture was a clear solution. A solution of BTC (0.35 eq.) in DCM (3 v) was added dropwise at 25-30° C. for 90 min. After addition, the reaction was stirred for 30 min at 25-30° C. A sample was taken to IPC showing residual compound 19 <3.0%. If the residual compound 19 >3.0%, an appropriate amount of BTC in DCM solution was added, the reaction was stirred for 30 min, sampled to get compound 19 <3.0%. (actual: 0.1 A % of compound 19)

H₂O (15 v) was charged into the reaction mixture at 25-30° C. The reaction was stirred for 60 min and phases were separated. The aqueous phase was extracted with DCM (5 v) once. The organic phases were combined, 5% of NaHCO₃ aq. (10 v) was charged into the reaction. The reaction was stirred for 60 min and phases were separated, the organic phase was collected. (total aqueous loss: 135 g). The organic phase was washed with H₂O (2*5 v) twice, and the organic phase was collected. Optionally, to detect the effect of contaminants, 9 g of (S, S) isomer of 101, 5 g of (R, R) isomer of 101, 4.4 g of (R, S) isomer of 101, and compound 19 (50 g. after spiking, the material left 1.2 A %) were charged into the organic phase, and stirred to get a clear solution (this step may be eliminated during the synthesis, and in this run these contaminants were removed in the final purification).

The solution was polish filtered with a microporous filter and the filtrate was collected into Reactor B. The organic phase was concentrated under vacuum with Jacket temperature kept at 38° C. to 3˜4 v and DCM was exchanged for iPrOAc (2*7 v) which had been previously filtered with a microporous filter. The reaction mixture was adjusted to 6 volumes with iPrOAc (previously filtered with a microporous filter). The inner temperature was kept at 38±1° C., and Form A crystal 0.5 wt % was added as a seed. The mixture was stirred for 40 min at 38±1° C.

Heptane (25 v) which had been previously filtered with a microporous filter was added dropwise at 38±1° C. for at least 2 hours. After addition, the reaction was stirred for 30 min, then cooled to 35±1° C., stirred for 5 min, then cooled to 32±1° C., stirred for 5 min, until cooled to 5±1° C., then stirred for at least another 13 hours.

The reaction was filtered, and the filter cake was washed with Heptane (2 v, which has been previously filtered with a microporous filter) to get a yellow solid, which was sampled by HPLC. The filter cake was dried at 40±5° C., P≤−0.08 MPa, for at least 16.0 hours. A sample was taken for LOD. Obtained 2.85 kg product compound 101, HPLC: 98.59 A %, yield: 86.9%, % de: 99.9%, % ee: 99.9%. The product was submitted to QC for release. (ML lost: 149 g).

LC-MS: (ES, m/z): [M+H]⁺ 540

¹H-NMR: (400 MHz, DMSO-d6, ppm): 60.84-0.91 (m, 4H), δ 1.38-1.95 (m, 12H), δ 2.08-2.10 (m, 1H), δ 2.68-2.77 (m, 2H), δ 3.19-3.25 (m, 3H), δ 3.43 (s, 3H), δ 4.25-4.28 (d, 1H), δ 7.01 (s, 1H), δ 7.19-7.21 (d 1H), δ 7.32 (s, 1H), δ 7.43-7.46 (t, 1H), δ 7.66-7.75 (m, 3H), δ 8.34 (s, 1H).

1. A method of producing compound 101, the method comprising the step:
a) reacting compound 19 with X—C(O)—Y, wherein X and Y are each leaving groups, to produce compound 101

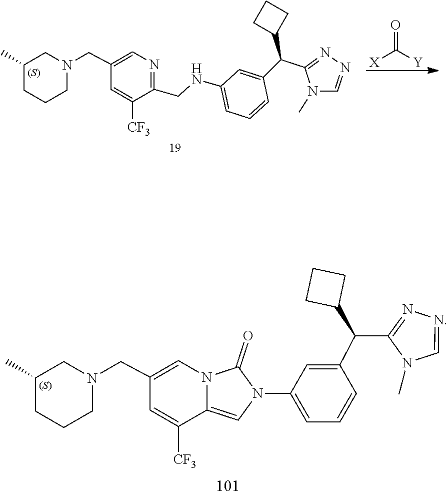

2. The method of claim 1, wherein X—C(O)—Y is phosgene or bis(trichloromethyl)carbonate.

3. The method of claim 1, wherein X—C(O)—Y is bis(trichloromethyl)carbonate.

4. The method of any one of claims 1-3, further comprising the step:
b) reacting compound 18 with compound 8 in the presence of a reducing agent to produce compound 19

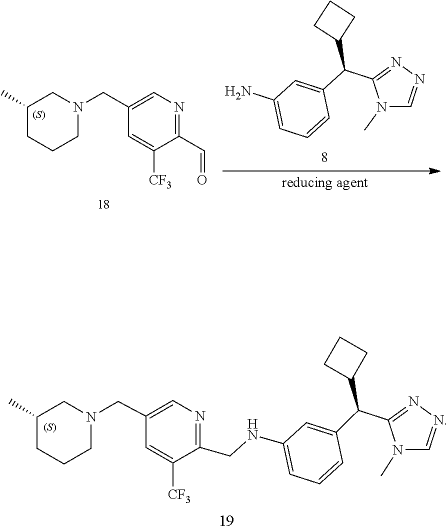

5. The method of claim 4, wherein the step b) reducing agent is a borohydride such as NaBH(OAc)₃ or NaBH₃CN.

6. The method of any one of claims 1-5, the method further comprising the steps:
c) reacting compound 14′ with compound 11 in the presence of a catalyst A to produce compound 15′ 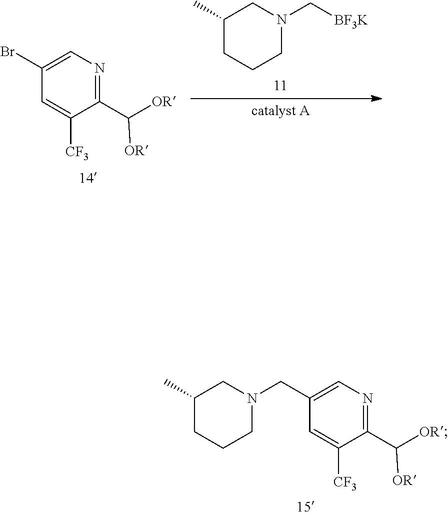
and
d) reacting compound 15′ with acid to produce compound 18 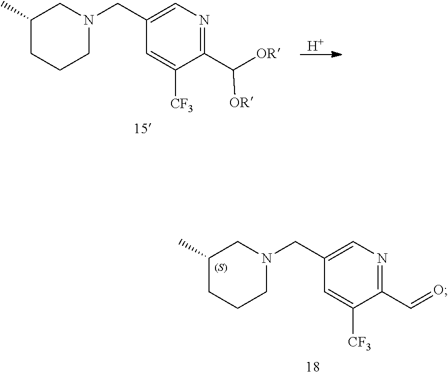
wherein each R′ is independently C₁₋₆ alkyl; or
both R′ groups are taken together as a C₁₋₆ alkylene; and
catalyst A is an organometallic catalyst.

7. The method of claim 6, wherein each R′ is methyl or ethyl, or both R′ groups taken together form —CH₂—CH₂— or —CH₂CH₂CH₂—.

8. The method of claim 7, wherein both R′ groups taken together form —CH₂—CH₂—.

9. The method of any one of claims 6-8, wherein catalyst A comprises a palladium atom, preferably wherein catalyst A is suitable for catalyzing a Suzuki reaction.

10. The method of claim 9, wherein catalyst A is formed from the combination of a Pd (II) salt (such as Pd(OAc)₂) and xphos.

11. The method of any one of claims 1-10, the method further comprising the steps:
e) separating the racemic mixture 6 by chiral Supercritical fluid chromatography (SFC) to obtain compound 7 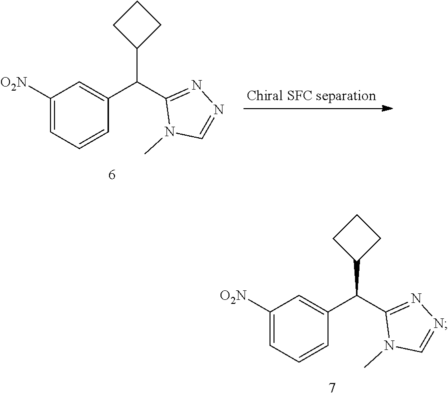 [SMILES: Cn1cnnc1C(c1cccc([N+](=O)[O-])c1)C1CCC1 ; Cn1cnnc1[C@@H](c1cccc([N+](=O)[O-])c1)C1CCC1]
and
f) reducing compound 7 to provide compound 8

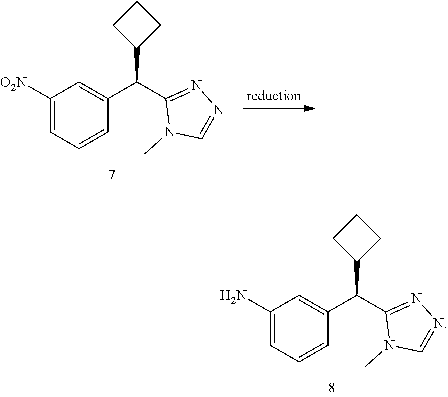 [SMILES: Cn1cnnc1[C@@H](c1cccc([N+](=O)[O-])c1)C1CCC1 ; Cn1cnnc1[C@@H](c1cccc(N)c1)C1CCC1]

12. The method of claim 11, wherein the step e) separating is performed on a cellulose-SC column.

13. The method of claim 12, wherein the step e) separating is performed with mobile phase A is CO₂, and mobile phase B comprises methanol, acetonitrile, and NH₃.

14. The method of claim 13, wherein mobile phase B is methanol:acetonitrile from 2:3 to 3:2 with 1-3 mM NH₃.

15. The method of any one of claims 11-14, wherein the step f) reducing is performed by catalytic hydrogenation, such as with hydrogen and a catalyst comprising palladium or platinum.

16. The method of any one of claims 1-15, the method further comprising the steps:
g) reacting compound 3 with methyl isothiocyanate to obtain compound 4 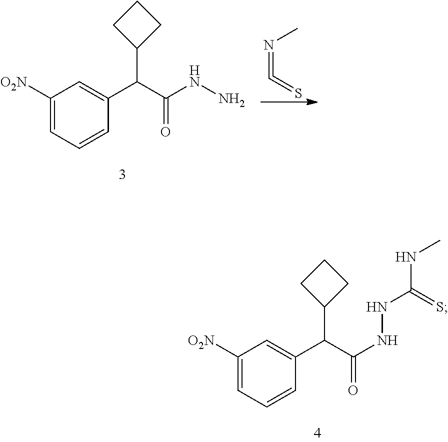
h) reacting compound 4 with base to provide compound 5 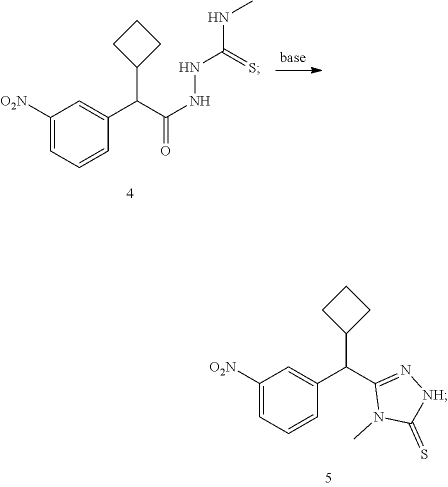 [SMILES: CNC(=S)NNC(=O)C(c1cccc([N+](=O)[O-])c1)C1CCC1 ; Cn1c(C(c2cccc([N+](=O)[O-])c2)C2CCC2)n[nH]c1=S]
and
i) reacting compound 5 with sodium nitrite and acid to produce compound 6

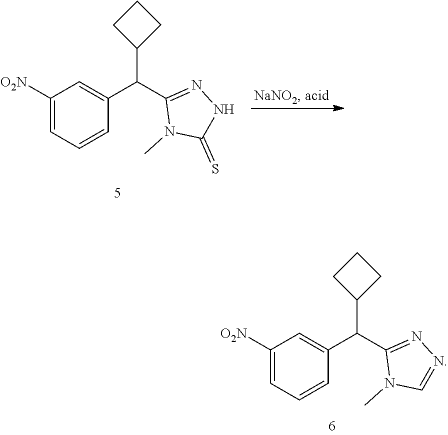 [SMILES: Cn1c(C(c2cccc([N+](=O)[O-])c2)C2CCC2)n[nH]c1=S ; Cn1cnnc1C(c1cccc([N+](=O)[O-])c1)C1CCC1]

17. The method of any of claims 1-16, wherein compound 101 is crystallized from a mixture of isopropyl acetate and heptane.

18. The method of any of claims 1-17, wherein compound 101 is at least 95% pure by HPLC.

19. An intermediate compound useful for the preparation of compound 101 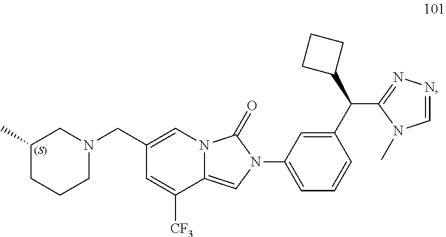
wherein the intermediate compound is selected from the group consisting of compounds 1-19 of schemes 1-3.

20. The intermediate compound of claim 19, having the structure

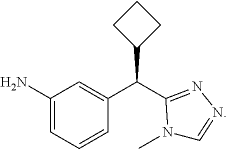 [SMILES: Cn1cnnc1[C@@H](c1cccc(N)c1)C1CCC1]

21. The intermediate compound of claim 19, having the structure

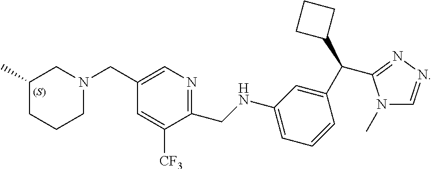

22. A method of producing an intermediate compound useful for the preparation of compound 101 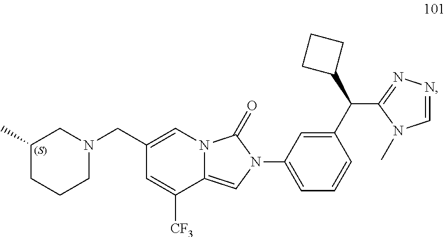
wherein the intermediate compound is selected from the group consisting of compounds 1-19 of schemes 1-3, the method comprising steps found in the examples.

23. A method of producing compound 8 with structure shown below, the method comprising the steps:
e) separating the racemic mixture 6 by chiral Supercritical fluid chromatography (SFC) to obtain compound 7 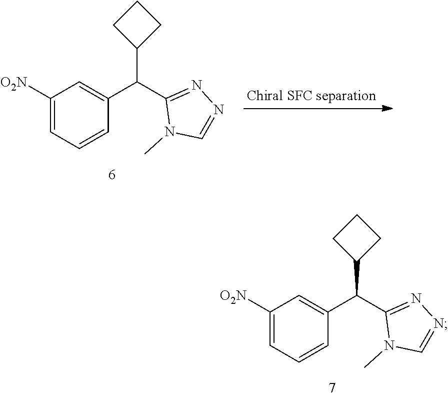 [SMILES: Cn1cnnc1C(c1cccc([N+](=O)[O-])c1)C1CCC1 ; Cn1cnnc1[C@@H](c1cccc([N+](=O)[O-])c1)C1CCC1]
and
f) reducing compound 7 to provide compound 8

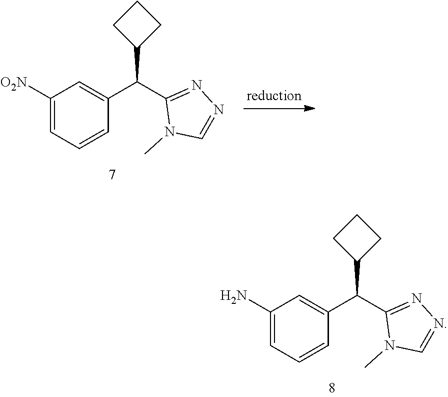 [SMILES: Cn1cnnc1[C@@H](c1cccc([N+](=O)[O-])c1)C1CCC1 ; Cn1cnnc1[C@@H](c1cccc(N)c1)C1CCC1]

24. The method of claim 23, wherein the step e) separating is performed on a cellulose-SC column.

25. The method of claim 24, wherein the step e) separating is performed with mobile phase A is CO₂, and mobile phase B comprises methanol, acetonitrile, and NH₃.

26. The method of claim 25, wherein mobile phase B is methanol:acetonitrile from 2:3 to 3:2 with 1-3 mM NH₃.

27. The method of any one of claims 23-26, wherein the step f) reducing is performed by catalytic hydrogenation, such as with hydrogen and a catalyst comprising palladium or platinum.

28. The method of any one of claims 23-27, the method further comprising the steps:
g) reacting compound 3 with methyl isothiocyanate to obtain compound 4 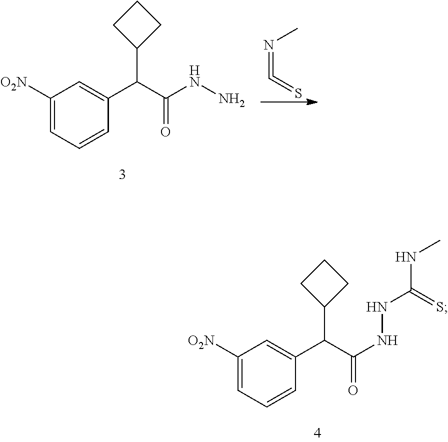
h) reacting compound 4 with base to provide compound 5 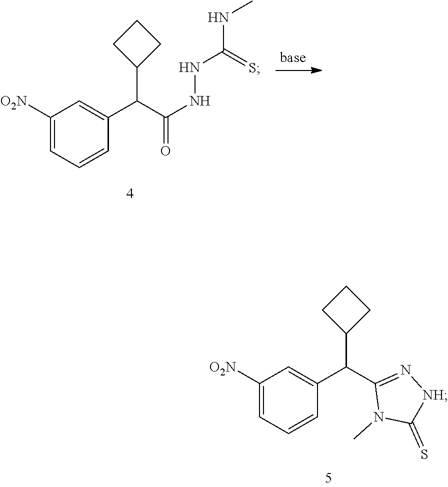 [SMILES: CNC(=S)NNC(=O)C(c1cccc([N+](=O)[O-])c1)C1CCC1 ; Cn1c(C(c2cccc([N+](=O)[O-])c2)C2CCC2)n[nH]c1=S]
and
i) reacting compound 5 with sodium nitrite and acid to produce compound 6 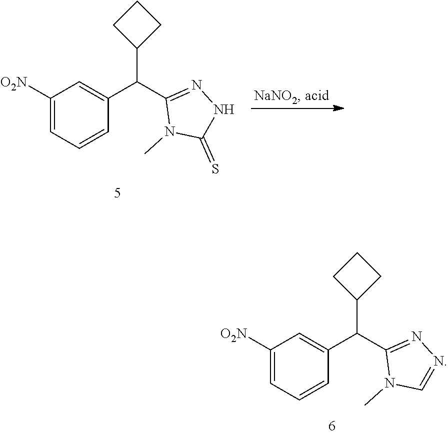 [SMILES: Cn1c(C(c2cccc([N+](=O)[O-])c2)C2CCC2)n[nH]c1=S ; Cn1cnnc1C(c1cccc([N+](=O)[O-])c1)C1CCC1]
